(19) United States
(12) Patent Application Publication
(10) Pub. No.: US 2004/0241656 A1
(43) Pub. Date: Dec. 2, 2004

(54) High-performance bio-tube for purifying DNA and method thereof

(76) Inventors: Ming-Shiung Jan, Hsin-Chu (TW); Chin-Horng Wang, Hsin-Chu City (TW)

Correspondence Address: BIRCH STEWART KOLASCH & BIRCH, PO BOX 747, FALLS CHURCH, VA, 22040-0747, US

(21) Appl. No.: 10/447,159

(22) Filed: May 29, 2003

(51) Int. Cl. C12Q 1/68; C07H 21/04; C12N 1/08

(52) U.S. Cl. 435/6; 435/270; 536/25.4

(57) ABSTRACT

A high-performance bio-tube for purifying DNA and a method thereof are provided. The high-performance bio-tube is provided with bio-particles immobilized therein. The bio-particles have particle sizes not more than 100 μm and is capable of binding DNA. A biological sample is added in the bio-tube and then a lysis buffer is added to form an admixture of the biological sample and the lysis buffer for lysing the cells to release DNAs. The admixture is maintained in the bio-tube in a period of time sufficiently to make the released DNAs binding to the bio-particles immobilized in the bio-tube. The remaining admixture in the bio-tube is directly discarded. Then, eluting DNAs bound to the bio-particles by feeding an elution solution in the bio-tube.

### BACKGROUND OF THE INVENTION

1. Field of the Invention

The present invention relates to an apparatus and method for purifying DNA from a biological sample, and more particularly to a bio-tube having bio-particles immobilized therein and a method thereof for purifying DNA.

2. Description of the Prior Art

The isolation of preparative amounts of biologically active nuclei acid molecules has been a vexing problem in molecular biology. This is especially the case with regard to isolation of DNA for use in recombinant methodologies where it is required to be in sufficiently pure form to be digestible by restriction endonuclease, to be a good substrate for polymerases and topoisomerases, and to be suitable for use as a transfection or transformation agent.

Over the years, many methods have been developed to isolate nuclei acid molecules. However, those method are typically tedious, require a high level of skill to perform, take extended periods of time to accomplish, require the processing of relatively large volumes of materials and often give variable results.

FIG. 1 shows a conventional purification process for isolating DNA from a human blood specimen. In step 11, a human blood specimen containing DNA, RNA, proteins and other cell components is added in a tube, and in step 12, a solution of phenol/chloroform/isoamyl alcohol is added in the tube to mix the human blood specimen. Then, in step 13, the mixture of human blood specimen and the solution of phenol/chloroform/isoamyl alcohol is centrifuged to give a sediment and a supernatant. The DNAs are maintained in the supernatant. Then, in step 14, transferring the supernatant containing DNAs in another tube by pipette. In step 15, adding a solution of sodium acetate/100% ethanol in the tube to mix the supernatant containing DNAs. In step 16, the mixture of the supernatant containing DNAs and the solution of sodium acetate/100% ethanol is centrifuged to give a sediment of DNA and a supernatant. In step 17, discarding the supernatant and collecting the sediment of DNA. Additional steps can be performed to further collect solid DNAs from the sediment. In step 18, a solution containing 70% ethanol is added to mix the sediment of DNA, and in step 19, the mixture is centrifuged to give a sediment of DNA and a supernatant. Repeating step 17, discarding the supernatant and collecting the sediment of DNA. The step 17 to step 19 can be repeated twice or more times to obtain purified DNAs.

The conventional purification process for DNA as shown in FIG. 1 is complicated, tedious, and requires a person of high-skilled level to manipulate the process to recover DNA at high-purified yield, and needs a large of working time.

FIG. 2 shows another conventional purification process for DNA utilizing the bonding properties of DNA to the surface of glass in the presence of a chaotropic agent. As shown in FIG. 2, in step 21, glass beads as a DNA-binding solid phase and NaI as a chaotropic agent for assisting binding of DNA are added in a tube. In step 22, a human blood specimen including DNA, RNA, proteins and other cell components is added in the tube to form an admixture of the human blood specimen and NaI. Then, in step 23, the admixture is incubated for about 5 minutes at room temperature to form a DNA-binding solid phase, i.e. the DNA-binding glass beads. In step 24, performing a centrifugation step to the admixture for isolating the DNA-binding glass beads from the admixture. In step 25, discarding the supernatant and maintaining the DNA-binding glass beads in the tube. In step 26, washing the DNA-binding glass beads with a solution containing 70% ethanol three times, and discarding the supernatant by pipette and maintaining the DNA-binding glass beads in the tube. Then, in step 27, adding TE buffer (10 mM Tris, 1 mM EDTA, pH 8.0) in the tube to incubate for about 2-3 minutes at 45° C. to elute DNAs from the glass beads. In step 28, performing a centrifugation step to the tube to give a supernatant of TE buffer containing DNAs. Finally, in step 29, transferring the TE buffer containing DNAs in another tube by pipette.

This conventional purification method of DNA shown in FIG. 2 is also complicated, tedious, and requires the person of high-skilled level to operate, and the auxiliary bio-agent, such as glass beads, is expensive, making the DNA purification process cannot be cost down.

Accordingly, it is an intention to develop an apparatus and method for DNA purification from a biological sample, which can alleviate the drawbacks of the conventional purification processes.

### SUMMARY OF THE INVENTION

It is one objective of the present invention to provide a high-performance bio-tube for purifying DNA and a method thereof, which can rapidly and easily purify DNA from a biological sample at a high yield.

It is another objective of the present invention to provide a high-performance bio-tube for purifying DNA and a method thereof, which permits a significant reduction in the level of skill and time required to produce isolated DNA.

It is a further objective of the present invention to provide a high-performance bio-tube with bio-particles immobilized therein, which is easily prepared and can attain cost down for a DNA purification process.

In order to achieve the above objectives of this invention, the present invention provides a high-performance bio-tube for purifying DNA from a biological sample and a method thereof. The high-performance bio-tube is provided with bio-particles immobilized therein. The bio-particles have particle sizes not more than 100 μm and are capable of binding DNA. A biological sample to be treated is added in the bio-tube, and then a lysis buffer is added in the bio-tube to form an admixture of the biological sample and the lysis buffer for lysing cells in the biological sample to release DNAs. The admixture is maintained in the bio-tube in a period of time sufficiently to make the released DNAs binding to the bio-particles. Then, the remaining admixture in the bio-tube is directly discarded. Finally, feeding a solution for eluting DNAs bound to the bio-particles in the tube, and collecting the eluted solution containing DNAs.

A simple and rapid process for purifying DNA from a biological sample, which without requiring an operator of high-skilled level and additional sample manipulations, is obtained, with the high-performance bio-tube of the present invention. Furthermore, the purification method with the present high-performance bio-tube permits the purified DNA to be used directly in cloning, sequence or other techniques.

### BRIEF DESCRIPTION OF THE DRAWINGS

The objectives and features of the present invention as well as advantages thereof will become apparent from the following detailed description, considered in conjunction with the accompanying drawings.

FIG. 1 shows a flow chart of a conventional DNA purification process;

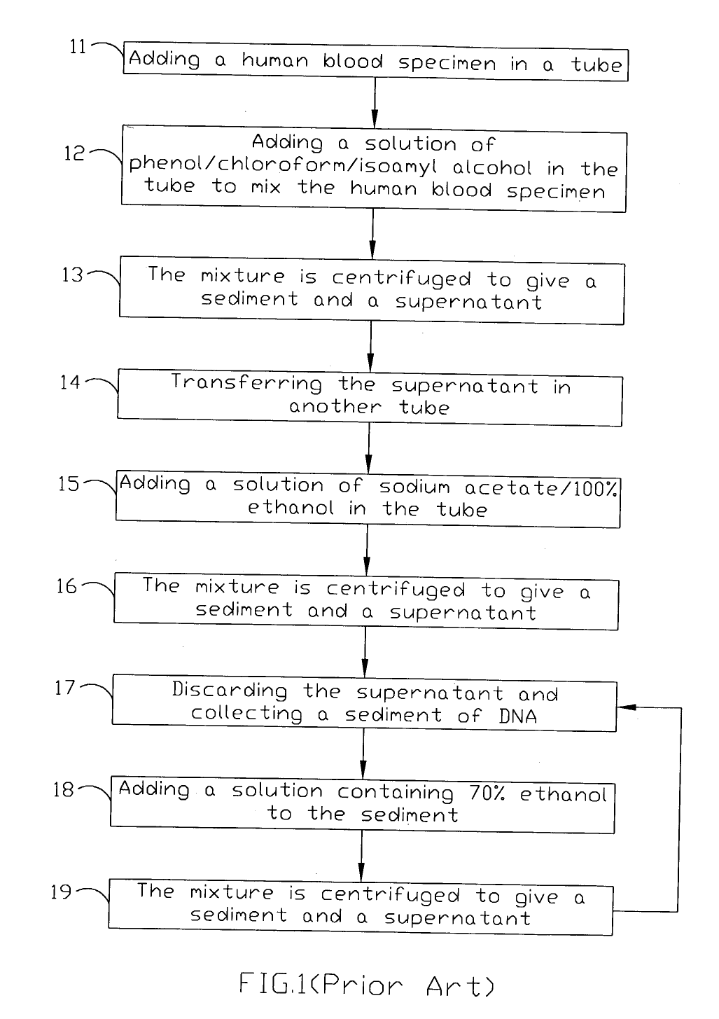

FIG. 2 shows a flow chart of another conventional DNA purification process;

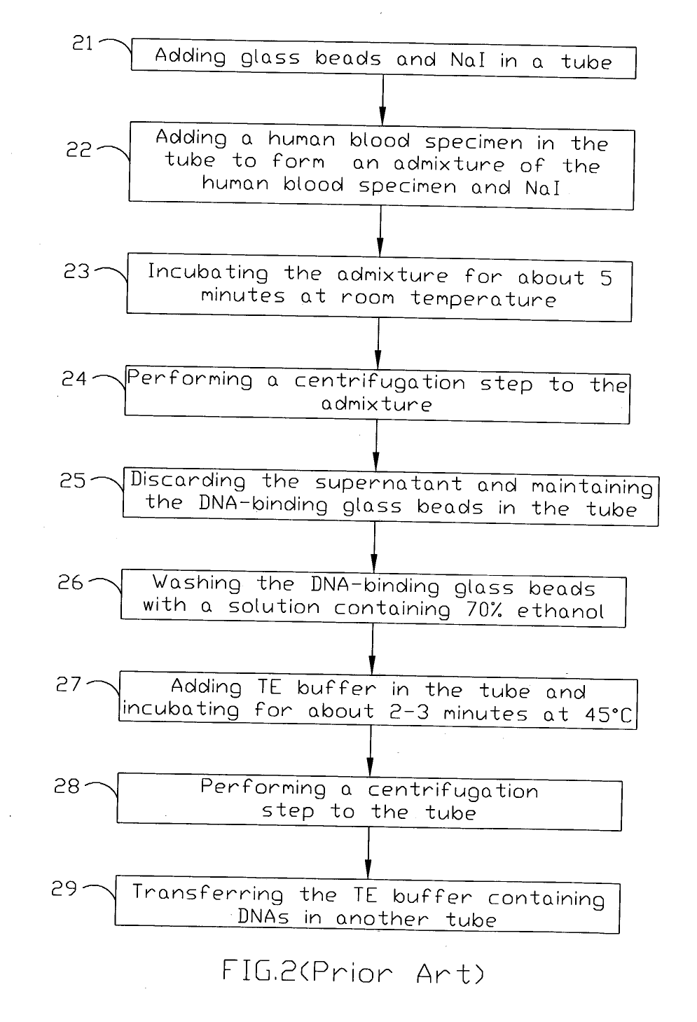

FIG. 3 is an exemplary perspective view of a high-performance bio-tube of the present invention; and

FIG. 4 is a flow chart of a DNA purification method utilizing the high-performance bio-tube of the present invention.

### DESCRIPTION OF THE EMBODIMENTS

DNA interacts with a solid phase surface in two ways. First, DNA interacts with the surface through hydrogen bonding between hydroxyl groups of DNA and surface components of the solid phase, such as surface hydroxyls. The second interaction is between the negatively charged phosphates of the DNA and positively charged elements of the solid phase surface. The hydrophilic and electropositive characteristics of the solid phase surface must be such as to allow binding of the DNA from a suspension of cellular components, a suspension of nucleic acid and other components, and to permit elution of the DNA from the solid phase material. Thus, it is desired to produce solid phase surfaces, which exhibit suitable hydrophilic and electropositive characteristics for DNA purification.

The present invention provides a high-performance bio-tube having bio-particles immobilized therein for recovery DNA from a biological sample. The bio-particles immobilized in the bio-tube of the present invention, by the material or modified surface thereof as mentioned above, exhibits sufficient hydrophilicity and sufficient electropositivity to bind DNA from cellular components and permit elution of the DNA from the immobilized bio-particles.

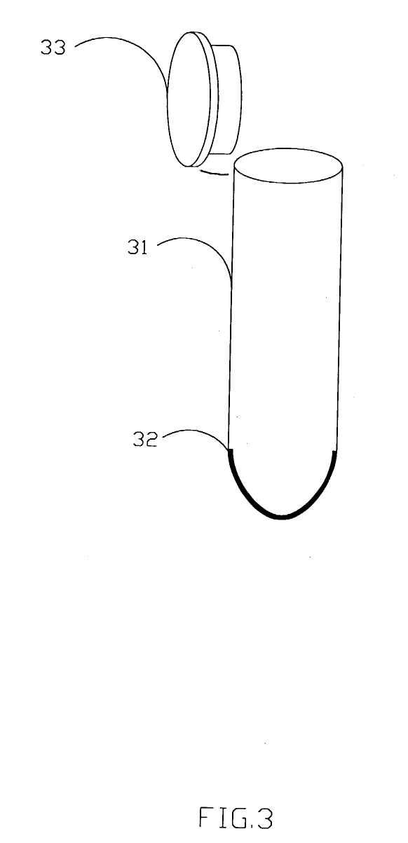

FIG. 3 is an exemplary perspective view of a high-performance bio-tube of the present invention. The high-performance bio-tube mainly includes a tube 31 and a thin film of bio-particles 32 immobilized in the inner bottom surface of the tube 31. A cap 33 is used to seal the tubes 31. The thin film of bio-particles 32 can be directly coated in the inner bottom surface of the tube 31 by a spray technique to form a solid phase support immobilized in the tube 31. The bio-particles 32 have particle sizes not more than 100 μm, and modified surfaces exhibiting sufficient hydrophilic and electropositive characteristics for binding DNA. On the surfaces of the bio-particles 32, hydrophilic characteristics can be achieved by the presence of groups that will attract water molecules. Suitable groups include —OH, —NH, —F, —H or groups with double-bonded oxygen such as carbonyl, sulfonyl or phosphonyl. Electropositive characteristics can be achieved by the presence of positively charged atoms. Suitable positively-charged atoms include Si, B or Al. In the present invention, the hydrophilic characteristics can be achieved by incorporation of the appropriate hydrophilic groups to modify the surfaces of the bio-particles 32, and the electropositive characteristics are achieved by incorporation of Si and other appropriate positively-charged atoms to modified the surfaces of the bio-particles 32. Besides, the bio-particles 32 of the present invention can be formed of silicon-containing material including boron, silicates, aluminum silicates, phosphosilicates, silica carbonyl, silica sulfonyl and silica phosphonyl. The hydrophilic characteristics can be achieved by incorporation of the appropriate hydrophilic groups to the silicon-containing material, and the electropositive characteristics can be achieved by incorporation of Si and other appropriate positively-charged atoms to the silicon-containing material.

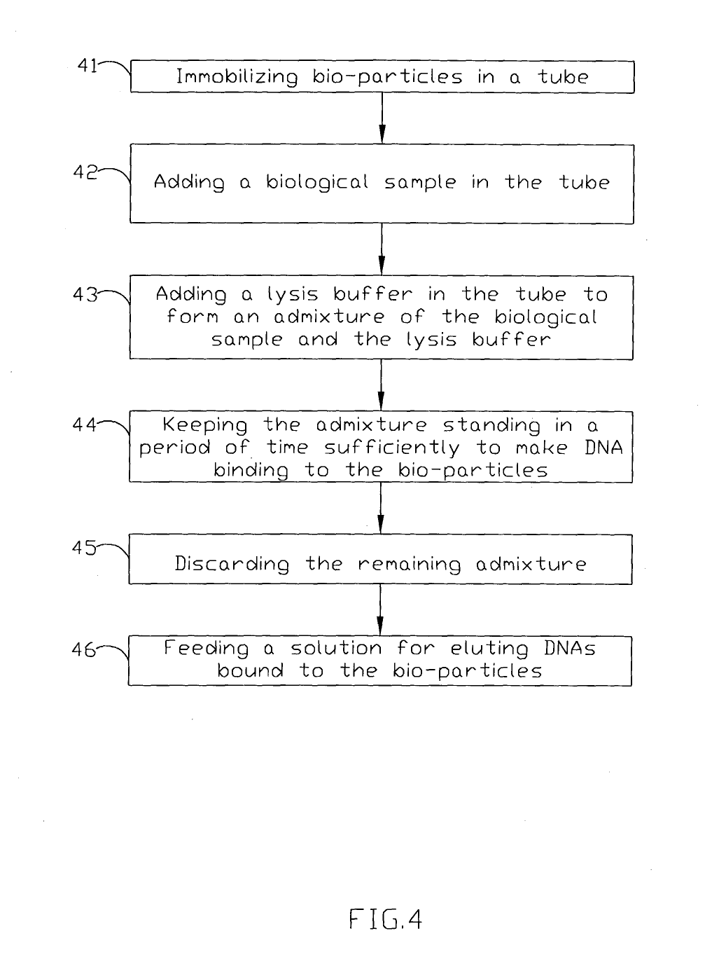

FIG. 4 shows a flow chart of a DNA purification method utilizing the high-performance bio-tube of the present invention. However, the present DNA purification method is described by reference to the following examples, which are offered by way of illustration and are not intended to limit the invention in any manner. In step 41, the high-performance bio-tube with bio-particles 32 immobilized therein is prepared. In step 42, a biological sample, for example clinical specimens such as of human blood, blood serum, phlegm, urine and the like, or biological specimens such as of cultured cells, cultured bacteria and the like, is added in the tube 31. Then, in step 43, adding a lysis buffer in the tube 31 to form an admixture of the biological sample and the lysis buffer to lyse cells in the biological sample to release DNA. The composition of the lysis buffer depends on the biological sample. For example, the lysis buffer for lysing cells of the human blood sample includes 20 mM Tris-HCl (pH 7.5), 150 mM NaCl, 1 mM Na₂EDTA, 1 mM EGTA, 1% 2.5 mM sodium pyrophosphate, 1 mM β-glycerophosphate, 1 mM Na₃VO₄ (sodium ortho-vanadate), 1 μg/ml leupeptin. The cell lysis conditions are well known, and a detailed description would not be described herein. Then, in step 44, keeping the admixture standing in the tube 31 in a period of time sufficiently to make released DNA binding to the bio-particles 32. For example, it is preferable about 10 minutes to stand the admixture of the human blood sample and the lysis buffer in the tube 31 to make released DNA binding to the bio-particles 32. Then, in step 45, since the DNA-binding bio-particles are immobilized in the tube 31, the remaining admixture is discarded by directly pouring out for isolating the DNA-binding bio-particles 32 from the admixture. The DNA-binding bio-particles 32 are still immobilized in the tube 31. Finally, in step 46, a solution is fed in the tube 31 with the DNA-binding bio-particles 32 immobilized therein to elute the DNAs from the bio-particles 32. The elution solution for the human blood sample can be water. Then, the eluted DNA-containing solution can be directly transferred by pipette or directly conduct a polymerase chain reaction (PCR) in the tube 31 to amplify the purified DNA to about a million fold for genetic tests or gene analyses. The elution solution containing DNA collected from step 46 is electrophoresed on an agarose gel. The band of visualized DNA recovered from the human blood by the present high-performance bio-tube is much brighter than that recovered from the human blood by the commercial-available kit. Moreover, the centrifugation step is omitted in the present DNA purification process and only fewer steps required. The DNA purification process utilizing the high-performance bio-tube of the present invention is simplified, easy operated, and shortens working time. In other words, the DNAs at a suitable purity for genetic tests or gene analyses can be rapidly and easily recovered by the present high-performance bio-tube without an artisan of high-skilled level.

The embodiments are only used to illustrate the present invention, not intended to limit the scope thereof. Many modifications of the embodiments can be made without departing from the spirit of the present invention.

What is claimed is:

1. A method of purifying DNA from a biological sample, comprising:
providing a tube;
immobilizing bio-particles in said tube, said bio-particles having particle sizes not more than 100 μm and capable of binding DNA;
adding a biological sample in said bio-tube;
adding a lysis buffer in said bio-tube to form an admixture of the biological sample and the lysis buffer for lysing cells in the biological sample to release DNA, and keeping the admixture standing in a period of time sufficiently to make the released DNA binding to said bio-particles;
discarding the remaining admixture; and
feeding a solution for eluting DNAs bound to said bio-particles in said tube, and collecting the eluted solution containing DNAs.

2. The method of claim 1, wherein said bio-particles are coated in said tube.

3. The method of claim 1, wherein said bio-particles have modified surfaces exhibiting sufficient hydrophlilic and electropositive characteristics for binding DNA.

4. The method of claim 2, wherein said bio-particles have modified surfaces exhibiting sufficient hydrophlilic and electropositive characteristics for binding DNA.

5. The method of claim 3, wherein the hydrophilic characteristic of the modified surfaces of said bio-particles is achieved by incorporation of hydrophilic groups.

6. The method of claim 3, wherein the electropositive characteristic of the modified surfaces of said bio-particles is achieved by incorporation of silicon and other positive-charged atoms.

7. The method of claim 4, wherein the hydrophilic characteristic of the modified surfaces of said bio-particles is achieved by incorporation of hydrophilic groups.

8. The method of claim 4, wherein the electropositive characteristic of the modified surfaces of said bio-particles is achieved by incorporation of silicon and other positive-charged atoms.

9. The method of claim 1, wherein said bio-particles are formed of a silicon-containing material.

10. The method of claim 9, wherein said bio-particles are coated in said tube.

11. The method of claim 9, wherein said bio-particles exhibit sufficient hydrophlilic and electropositive characteristics for binding DNA.

12. The method of claim 11, wherein the hydrophilic characteristic of said bio-particles is achieved by incorporation of hydrophilic groups.

13. The method of claim 11, wherein the electropositive characteristic of said bio-particles is achieved by incorporation of silicon and other positive-charged atoms.

14. The method of claim 1, wherein the composition of said lysis buffer is based on the biological sample to be treated.

15. The method of claim 1, wherein said biological sample is human blood.

16. The method of claim 15, wherein the time for making the released DNAs binding to said bio-particles is about 10 minutes.

17. The method of claim 16, wherein the solution for eluting DNAs bound to said bio-particles is water.

18. A high-performance bio-tube for purifying DNA from a biological sample, comprises:
a tube; and
a thin film of bio-particles immobilized in said tube, said bio-particles having particle sizes not more than 100 μm and capable of binding DNA.

19. The high-performance bio-tube of claim 18, wherein said bio-particles exhibit sufficient hydrophlilic and electropositive characteristics for binding DNA.

20. The high-performance bio-tube of claim 18, wherein said bio-particles are formed of a silicon-containing material.
